(19) United States
(12) Patent Application Publication
(10) Pub. No.: US 2017/0100025 A1
(43) Pub. Date: Apr. 13, 2017

(54) CLEANING AND/OR DISINFECTING DEVICE

(71) Applicant: OLYMPUS WINTER & IBE GMBH, Hamburg (DE)

(72) Inventor: Michael LENGSFELD, Hamburg (DE)

(73) Assignee: OLYMPUS WINTER & IBE GMBH, Hamburg (DE)

(21) Appl. No.: 15/384,844

(22) Filed: Dec. 20, 2016

Related U.S. Application Data
(63) Continuation of application No. PCT/EP2015/063314, filed on Jun. 15, 2015.

(30) Foreign Application Priority Data
Jun. 23, 2014 (DE) 102014211961.8

(51) Int. Cl. A61B 1/12; A61L 2/18; A61L 2/24; B08B 3/04

(52) U.S. Cl. CPC A61B 1/123; B08B 3/04; A61L 2/18; A61L 2/24; A61L 2202/14; A61L 2202/17; A61L 2202/24

(57) ABSTRACT

A device for cleaning and/or disinfecting medical equipment. The device including: a storage container in which a liquid cleaning agent is provided; a metering pump coupled fluidically to the storage container, wherein the metering pump is configured for delivering cleaning agent from the storage container to provide cleaning and/or rinsing liquid for cleaning and/or disinfecting the medical equipment; and a calibrating device is fluidically coupled to the metering pump, the calibrating device comprises a measuring column, wherein the measuring column provides a calibration volume for calibrating a delivery rate of the metering pump.

### CROSS-REFERENCE TO RELATED APPLICATION

The present application is a continuation of PCT/EP2015/063314 filed on Jun. 15, 2015, which is based upon and claims the benefit to DE 10 2014 211 961.8 filed on Jun. 23, 2014, the entire contents of each of which are incorporated herein by reference.

### BACKGROUND

Field

The present application relates to a cleaning and/or disinfecting device for cleaning and/or disinfecting medical equipment, such as endoscopes, comprising a metering unit which is coupled fluidically to a storage container in which a liquid cleaning agent is present, wherein the metering unit is configured for delivering the cleaning agent from the storage container to provide cleaning and/or rinsing liquid for cleaning and/or disinfecting the medical equipment. The present application also relates to a method for operating a cleaning and/or disinfecting device.

Prior Art

High demands are placed on the reprocessing of medical equipment, such as surgical instruments including endoscopes. After the surgical instruments are used, they are disinfected and/or cleaned in a cleaning and/or disinfecting device. One suitable reprocessing apparatus is for example known under the designation of ETD 3 by the manufacturer Olympus Medical Systems, wherein the abbreviation ETD stands for Endo Thermo Disinfector.

Reprocessing surgical instruments typically comprises the steps of: washing, disinfecting and drying. One or two washing or pre-washing stages often precede disinfection. This is followed by rinsing stages with clear water and drying stages. To wash and/or disinfect the medical equipment, one or more chemicals that have a cleaning and/or disinfecting effect are added to a cleaning and/or rinsing liquid.

In the context of the present description, chemicals or chemical mixtures will generally be referred to as cleaning agents.

To produce the cleaning and/or rinsing liquid, the cleaning agent typically available in liquid form is additionally diluted with water. The cleaning agent is metered by a metering pump. This ensures that the right amount of cleaning agent is added to the cleaning and/or rinsing liquid.

Both overdosing and underdosing are capable of negatively affecting the results of cleaning. If the prescribed dose is too low, an insufficient cleaning or disinfecting effect may be expected. A dose that is too high is uneconomical on the one hand and, on the other hand, may lead to unnecessary wear of the medical equipment.

To ensure that the right amount of cleaning agent is provided to produce the cleaning and/or rinsing liquid, the metering pump of a cleaning and/or disinfecting device is regularly tested and calibrated. However for the operator, this service means a loss of machine time, and also the additional costs of employing the service technicians.

### SUMMARY

On the basis of such prior art, an object is to present a cleaning and/or disinfecting device, as well as a method for operating a cleaning and/or disinfecting device that is/are economical, wherein the complexity of the design can also be minimized

This object can be achieved by a cleaning and/or disinfecting device for cleaning and/or disinfecting medical equipment, such as endoscopes, comprising a metering unit which is coupled fluidically to a storage container in which a liquid cleaning agent is present, wherein the metering unit is configured for delivering cleaning agent from the storage container to provide a cleaning and/or rinsing liquid for cleaning and/or disinfecting the medical equipment.

Such devices and methods consider that calibrating a metering unit in a cleaning and/or disinfecting device with a measuring column is sufficiently reliable and precise, wherein this solution for calibration can also be realized with a simple design. A measuring column can be easily integrated into a cleaning and/or disinfecting device such that neither the user nor the service technician comes into direct contact with the possibly aggressive cleaning agent while calibration is being performed. The cleaning and/or disinfecting device can furthermore be very robust, i.e., not very error-prone. It can be readily calibrated by personnel who have not been technically trained. It is also possible for the calibration of the delivery rate or delivery amount of the metering unit to be fully automated by the unit.

The cleaning and/or disinfecting device can be developed such that a control and/or regulating apparatus is available that is set up:
to control the metering unit such that it delivers cleaning agent from the supply container into the calibration volume such that the initially empty calibration volume is completely filled,
to capture a value of an operating parameter of the metering unit that characterizes a delivery rate of the metering unit, or a delivery volume of cleaning agent delivered by the metering unit, and
to calibrate the metering unit by assigning a delivery rate calculated from the time required to fill the calibration volume and the size of the calibration volume, or by assigning a size of the calibration volume as a delivery volume to the captured value of the operating parameter.

The cleaning and/or disinfecting device can be developed such that the metering unit comprises a self-priming metering pump, such as a peristaltic pump, and/or an impeller meter, wherein the control and/or regulating apparatus is configured to capture a rotary speed of the metering pump, and/or a rotary speed of the impeller meter as an operating parameter of the metering unit while delivering cleaning agent into the calibration volume, and is configured to calibrate the metering unit in that the rotary speed captured while the cleaning agent is being delivered is assigned a calculated delivery rate of the metering unit by means of the size of the calibration volume and the time required to fill the calibration volume.

Furthermore, the metering unit can comprise i a self-priming metering pump, such as a peristaltic pump, and/or an impeller meter, wherein the control and/or regulating apparatus is configured to capture a number of rotations and/or cycles of the metering pump, and/or a number of rotations and/or cycles of the impeller meter, as an operating parameter of the metering unit during the delivery of cleaning agent into the calibration volume, and is configured to perform a calibration of the metering unit by assigning a volume of the calibration volume as the delivery volume to the number of rotations and/or cycles cumulatively captured during the delivery of the cleaning agent.

The cleaning agent can be metered by operating a metering pump at a specific rotary speed for a specific time. It is likewise possible to adjust the amount of cleaning agent to be metered by means of a number of rotations or cycles of a metering pump, such as a peristaltic pump. If the metering pump is for example a piston or diaphragm pump and not a rotating pump, the delivery rate can be controlled by a pump frequency or number of piston strokes or pump cycles. Alternatively or in addition, it is possible to control the metering pump with feedback, measure its delivery rate with the assistance of an impeller meter, and control the metering pump based on this value. Instead of an impeller meter, any other form of mass flowmeter can also be used. Independent of whether the metered amount of cleaning agent is controlled directly using an operating parameter of the metering pump such as its rotary speed, or by feedback using an impeller meter, it is easily and economically feasible according to the cited embodiment to optionally calibrate the pump itself or the employed mass flowmeter.

Furthermore, the cleaning and/or disinfecting device can be configured such that a first fill level sensor and a second fill level sensor are on the measuring column, wherein the first fill level sensor is configured to capture a first level in the measuring column that corresponds to a complete emptying of the calibration volume, wherein the second fill level sensor is configured to capture a second level in the measuring column that corresponds to a complete filling of the calibration volume, wherein the control and/or regulating apparatus is furthermore configured:
to capture a first value of the operating parameter of the metering unit when a fill state in the measuring column is determined by the first fill level sensor that corresponds to or exceeds the first level,
to capture a second value of the operating parameter of the metering unit when a fill state in the measuring column is determined by the second fill level sensor that corresponds to or exceeds the second level,
to determine a value for the operating parameter that characterizes the delivery amount needed to completely fill the calibration volume from the difference between the first and second value.

The measuring column, such as the calibration volume within the measuring column, can extend between a geodetically lower point at which the first fill level sensor is located and a geodetically higher point at which the second fill level sensor is located. For example, the measuring column can comprise a substantially cylindrical calibration volume, the direction of its longitudinal axis extending in the direction of gravity.

The cited embodiment allows simple and precise measurement of the fill level in the calibration volume with a simple design. A meniscus, i.e., a boundary surface between a liquid level and the air column lying above, can easily be captured by the first, or respectively the second, fill level sensor. It is accordingly possible to capture a fill state of the measuring column with simply designed means.

According to another embodiment, the control and/or regulating apparatus can be configured to lower the fill state of the measuring column below the level of the first level before a cleaning agent is delivered from the supply container into the calibration volume.

This measure advantageously improves the precision of calibration. Lowering the level of the measuring column below the level of the first level ensures that the calibration volume is filled by the metering unit continuously with cleaning agent once the first level is reached. During the entire filling process, the metering pump accordingly delivers the cleaning agent in only one direction, that is, into the calibration volume. The calibration error which is analogously known as “backlash” in mechanical systems is advantageously avoided.

Such object is further achieved by a method for operating a cleaning and/or disinfecting device according to one or more of the cited embodiments, wherein the method is developed in that the cleaning and/or disinfecting device is calibrated with the calibration device.

In such method, the calibration of the metering unit can be performed fully automated, such that the operator can dispense with employing a technician. This helps decrease operating costs and reduce machine downtimes. Furthermore, it is easily possible to perform the calibration method during times in which a service technician is normally not available and the cleaning and/or disinfecting device is not being intensively used. For example, it is possible to perform calibration on holidays or at nights. Regularly performing calibration furthermore eliminates the technical problem of pump performance drift which frequently arises in metering pumps over the course of time.

According to one embodiment, the method comprises the steps of:

controlling the metering unit such that it delivers cleaning agent from the supply container into the calibration volume such that the initially empty calibration volume is completely filled,
determining a value of an operating parameter of the metering unit that characterizes a delivery rate of the metering unit, or a delivery volume of cleaning agent delivered by the metering unit, and
calibrating the metering unit by assigning a delivery rate calculated from the time required to fill the calibration volume and the size of the calibration volume, or by assigning a size of the calibration volume as a delivery volume to the captured value of the operating parameter.

The method for operating the cleaning and/or disinfecting device can include the metering unit comprising a self-priming metering pump, such as a peristaltic pump, and/or an impeller meter, wherein a rotary speed of the metering pump, and/or a rotary speed of the impeller meter, is captured as an operating parameter of the metering unit while delivering cleaning agent into the calibration volume, and the metering unit is calibrated in that the rotary speed determined while the cleaning agent is being delivered is assigned a calculated delivery rate of the metering unit by means of the size of the calibration volume and the time required to fill the calibration volume.

Furthermore, the metering unit can comprise a self-priming metering pump, such as a peristaltic pump, and/or an impeller meter, wherein a number of rotations and/or cycles of the metering pump, and/or a number of rotations and/or cycles of the impeller meter, is captured as an operating parameter of the metering unit during the delivery of cleaning agent into the calibration volume, and a calibration of the metering unit is performed by assigning a volume of the calibration volume as the delivery volume to the number of rotations and/or cycles cumulatively captured during the delivery of the cleaning agent.

In another embodiment, the method can include providing a first fill level sensor and a second fill level sensor on the measuring column, wherein the first fill level sensor is configured to capture a first level in the measuring column that corresponds to a complete emptying of the calibration volume, and wherein the second fill level sensor is configured to capture a second level in the measuring column that corresponds to a complete filling of the calibration volume, wherein:
a first value of the operating parameter of the metering unit is captured when a fill state in the measuring column is determined by the first fill level sensor that corresponds to or exceeds the first level,
a second value of the operating parameter of the metering unit is captured when a fill state in the measuring column is determined by the second fill level sensor that corresponds to or exceeds the second level, and
a value for the operating parameter that characterizes the delivery amount needed to completely fill the calibration volume is determined from the difference between the first and second value.

The method can further include the fill state of the measuring column being lowered below the level of the first level before cleaning agent is delivered from the supply container into the calibration volume.

Same or similar advantages pertaining to the method for operating the cleaning and/or calibration device pertain to the cleaning and/or calibration device in a same or similar way.

In the cleaning and/or calibration device, the measuring column can be substantially cylindrical, wherein the calibration volume within the measuring column extends in a direction of the longitudinal axis of the measuring column This direction of the longitudinal axis can be oriented parallel to the direction of gravity. Alternatively, the measuring column can be realized by a wound tube. This can extend i from a geodetically lower point to a geodetically higher point, wherein a fill level sensor is provided at these two endpoints of the calibration volume. The calibration device can be provided for use in a cleaning and/or disinfecting device. The aforementioned aspects advantageously relate to all the cited embodiments.

Further features will become apparent from the description of embodiments together with the claims and the included drawings. Embodiments can fulfill individual characteristics or a combination of several characteristics.

### BRIEF DESCRIPTION OF THE DRAWINGS

Exemplary embodiments are disclosed herein with reference to the drawings, without limiting the general inventive idea, wherein relative to all of the details not described in more detail in the text, reference is expressly made to the drawings, in which:

FIG. 1 illustrates a schematic cleaning and/or disinfecting device,

FIG. 2 illustrates a simplified flowchart of a method for operating a cleaning and/or disinfecting device.

In the drawings, the same or similar types of elements and/or parts are provided with the same reference numbers so that a re-introduction is omitted.

### DETAILED DESCRIPTION

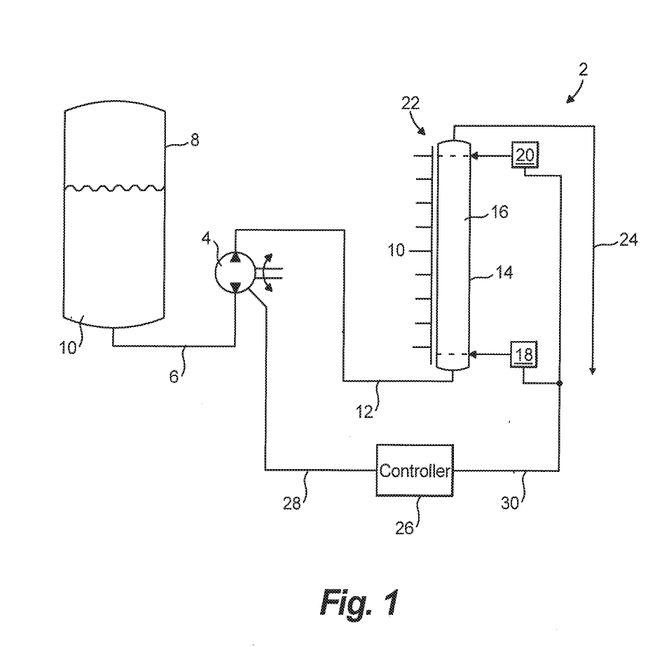

FIG. 1 schematically illustrates a cleaning and/or disinfecting device with a calibration device 2 for cleaning and/or disinfecting medical equipment. The cleaning and/or disinfecting device furthermore comprises a cleaning chamber (not shown) for accommodating the medical equipment during the rinsing or cleaning process. The medical equipment is for example surgical instruments, such as endoscopes. The cleaning and/or disinfecting device comprises a metering unit 4. According to the exemplary embodiment shown in FIG. 1, the metering unit can be a self-priming metering pump such as a peristaltic pump. A possible metering pump can be a rotating pump, piston pump or diaphragm pump. The metering unit 4 is fluidically coupled to a supply container 8 by means of a feedline 6. For example, the metering unit 4 is directly connected to the supply container 8 by means of a pipeline. The supply container 8 is provided for receiving a liquid cleaning agent 10. The cleaning agent is a cleaning chemical suitable for cleaning and/or disinfecting surgical instruments, such as endoscopes.

The metering unit 4 is furthermore fluidically coupled to a measuring column 14 by means of a delivery line 12. A pipeline for example is also suitable for this purpose that directly connects the metering unit 4 to the measuring column 14.

The metering unit 4 is not exclusively provided for delivering cleaning agent into the measuring column 14. The metering unit 4 can be connected to at least one other delivery line, which is not shown in FIG. 1, for operating the cleaning and/or disinfecting device. With the assistance of a delivery line 24, it is possible to supply cleaning agent 10 to the cleaning and/or disinfecting compartment (not shown) of the cleaning and/or disinfecting device such that a cleaning and/or rinsing liquid can be produced.

The measuring column 14 comprises a calibration volume 16 which extends between a first fill level sensor 18 and a second fill level sensor 20 in the interior of the measuring column 14. The size of the calibration volume 16 can be read from a schematically represented scale 22 and can be changed or respectively adjusted by for example vertically shifting the first and/or second fill level sensor 18, 20. The size of the calibration volume 16 is known. The measuring column 14 can be a cylindrical column with a circular cross section that for example is produced from glass or transparent plastic. Alternatively, a wound tube can be provided as the measuring column which extends between a geodetically lower point and a geodetically higher point. The first fill level sensor 18 is at the lower point, whereas the second fill level sensor 20 is at the higher of the two points. The measuring column 14 is furthermore connected to the delivery line 24 through which the cleaning agent 10 is delivered to the cleaning and/or disinfecting chamber.

The metering unit 4, which can be a peristaltic pump, can be calibrated in terms of its delivery rate by capturing the time required by the peristaltic pump to completely fill the calibration volume 16 with cleaning agent 10 starting from the known value of the calibration volume 16. To the extent that the metering unit 4 is provided with a mass flowmeter such as an impeller meter, the delivery rate of the pump can be measured and calibrated with the mass flowmeter by calibrating the impeller meter in terms of its rotary speed, for example. The delivery rate can be regulated with feedback through a mass flowmeter.

Calibration, for example based on the known value of the calibration volume, uses the captured time needed by the metering unit 4 to deliver cleaning agent 10 from the supply container 8 to the measuring column 14 and completely fill the calibration volume. The delivery rate calculated in this manner is assigned to the captured operating parameter such as the rotary speed of the pump.

Furthermore it is provided that, for example, a number of rotation cycles of the metering pump of the metering unit 4 is, or respectively are, captured that is/are necessary to completely fill the calibration volume. Based on the volume of the calibration volume, it is accordingly possible to determine a delivery amount per rotation or cycle of the measuring unit 4, or respectively the metering pump. There is a corresponding assignment to the unit of this operating parameter of the metering unit. In other words, optionally a calculated delivery rate is determined at a specific rotary speed of the metering pump, or a delivery amount, or respectively a delivered volume per cycle or per rotation of the metering pump is determined, and the metering unit 4 is calibrated with this value.

An operating parameter of the metering unit 4 is understood to include a rotary speed of the metering pump over a certain delivery time, a number of rotations that are necessary to fill the calibration volume, or a number of cycles that are necessary to completely fill the calibration volume.

To calibrate the metering unit 4, it is connected by a control and data line 28 to a control and/or regulating apparatus 26. The first fill level sensor 18 and the second fill level sensor 20 are also connected by a data line 30 to the control and/or regulating apparatus 26. The control and/or regulating apparatus 26 is configured to control the metering unit 4 such that the cleaning agent 10 is delivered through the feedline 6 from the supply container 8 and through the delivery line 12 into the measuring column 14. Furthermore, the control and/or regulating unit 26 is configured to read out at least one operating parameter such as the rotary speed of a peristaltic pump of the metering unit 4, or the rotary speed of an impeller meter, via the control and data line 28.

A first level is captured with the assistance of the first fill level sensor 18, for example by capturing a meniscus between a liquid/gas boundary surface. The boundary surface between the liquid cleaning agent 10 in the calibration volume 16 and an air column above can be captured. The same holds true for the second fill level sensor 20. The first and the second fill level sensor 18, 20 are for example optical sensors that capture the meniscus of the boundary surface in an optical manner, for example by measuring absorption or reflection.

A first level discernible by the first fill level sensor 18 corresponds to a fill state of the measuring column 14 when the calibration volume 16 is completely empty. A second level which is determined by the second fill level sensor 20 corresponds to a fill state of the measuring column 14 when the calibration volume 16 is completely full.

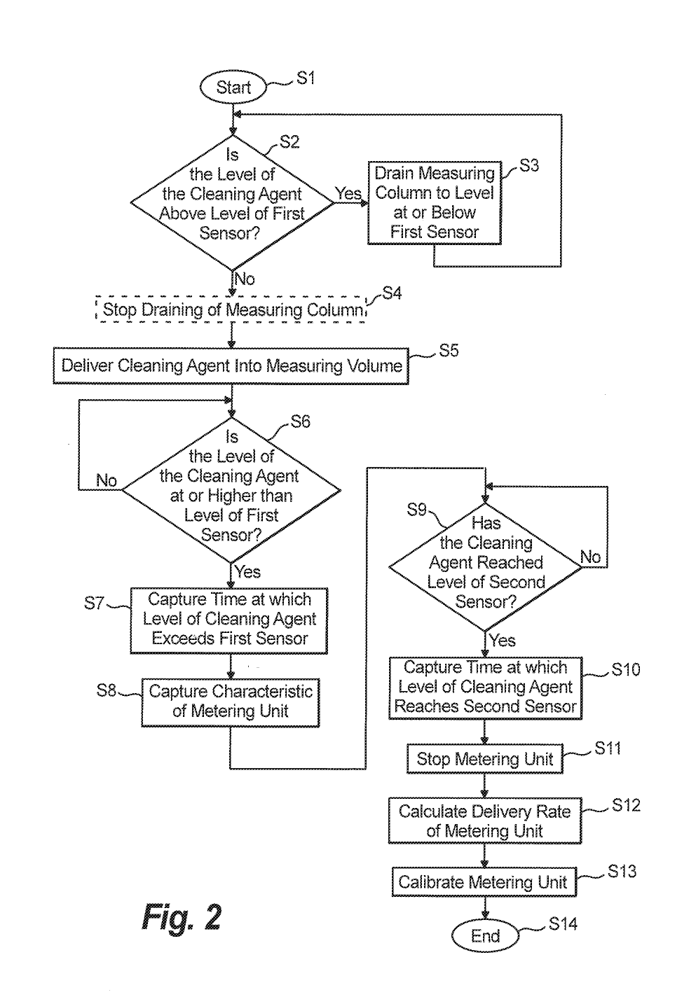

An example of the calibration of the metering unit 4, which is performed in the context of a method for operating the cleaning and/or disinfecting device 3, will be explained below with reference to the simplified flowchart in FIG. 2.

Furthermore, an example of the calibration of the metering unit 4 will be explained with reference to the instance in which a rotary speed of a metering pump or an impeller meter is captured for the time needed to fill the calibration volume 16. It is also possible to instead capture a number of rotations or cycles needed by a metering pump to completely fill the calibration volume 16.

After the start (step S1) of the calibration of the metering unit 4, the first fill level sensor 18 is initially queried. It is accordingly determined whether the fill level of the cleaning agent 10 in the measuring column 14 is above or below the first liquid level (step S2). If the liquid level in the measuring column 14 is above the first level measured by the first fill level sensor 18, it means that the calibration volume 16 is not completely empty. To establish the initial conditions defined for calibrating the metering unit 4, first a pumping process is initiated in which the calibration volume 16 is completely drained. To this end, the metering unit 4 such as a metering pump is controlled by the control and/or regulating unit 26 such that cleaning agent 10 is delivered by the metering unit via the feedline 6 back into the supply container 8 (step S3), or alternately cleaning agent 10 is discarded via a drain line (not shown).

During this reverse operation of the metering unit 4, the first fill level sensor 18 is continuously queried (step S2). If it is found that the liquid level within the measuring column 14 has dropped below the first level, the pumping-off process is stopped (step S4).

In the simplified flowchart in FIG. 2, step S4 is depicted with a dashed line since this step can be discarded in the event that the liquid level is already below the first level from the start. In such an initial situation, the method does not follow the path via step S3, but rather goes directly from step S2 to step S5.

In step S5, cleaning agent 10 is delivered by the metering unit 4 starting from the supply container 8 into the calibration volume 16. The first fill level sensor 18 is queried permanently such that it is discernible when the fill level within the measuring column 14 exceeds the first level (step S6). If this is the case, the time at which the first level is exceeded is captured. For example, the value for this first time is saved in a memory of the control and/or regulating apparatus 26, or a timer is started (step S7). Furthermore, a capturing of at least one operating parameter of the metering unit 4 takes place during the subsequent time span while the calibration volume 16 is being filled. For example, the rotary speed of a peristaltic pump or an impeller meter is captured during this time (step S8).

Directly afterward, the second fill level sensor 20 is permanently queried, which indicates whether the second level has been reached within the measuring column 14 (step S9). If the cleaning agent 10 reaches this second level, the calibration volume 16 is completely filled. The time is then recorded at which the fill level reaches or exceeds this second level. The value for this second time can in turn be saved in a memory of the control and/or regulating apparatus 26. If a timer was started at the first time, it is stopped (step S10).

Then the metering unit 4 is stopped (step S11), and a delivery rate of the metering unit 4 is calculated, or a measured delivery rate is calculated using the known volume of the calibration volume 16 and the difference in time between activating the first fill level sensor 18 and second fill level sensor 20 (step S12). The metering unit 4 is subsequently calibrated, such as by assigning the value of the calculated delivery rate of the metering unit 4 to the value of the at least one operating parameter captured while delivering the cleaning agent 10, such as the rotary speed (step S13). The same holds true for a calibration of a mass flowmeter such as an impeller counter. Finally, the calibration method terminates in step S14.

According to other exemplary embodiments, it is possible to perform the aforementioned method for different operating parameters of the metering unit 4. For example, the delivery rate of a metering pump is calibrated by determining the delivery rate for different rotary speeds.

The method for calibrating a metering unit 4 in a cleaning and/or disinfecting device can be performed fully automated. Advantageously, manual calibration of the metering unit 4 is hence unnecessary.

While there has been shown and described what is considered to be preferred embodiments, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### REFERENCE NUMBER LIST

2 Calibration device
4 Metering unit
6 Feedline
8 Supply container
10 Cleaning agent
12 Delivery line
14 Measuring column
16 Calibration volume
18 First level sensor
20 Second level sensor
22 Scale
24 Delivery line
26 Control and/or regulating apparatus
28 Control and data line
30 Data line

What is claimed is:

1. A device for cleaning and/or disinfecting medical equipment, the device comprising:
a storage container in which a liquid cleaning agent is provided;
a metering pump coupled fluidically to the storage container, wherein the metering pump is configured for delivering cleaning agent from the storage container to provide cleaning and/or rinsing liquid for cleaning and/or disinfecting the medical equipment; and
a calibrating device is fluidically coupled to the metering pump, the calibrating device comprises a measuring column, wherein the measuring column provides a calibration volume for calibrating a delivery rate of the metering pump.

2. The device according to claim 1, further comprising a control and/or regulating apparatus configured to:
control the metering pump such that it delivers cleaning agent from the supply container into the calibration volume such that an initially empty calibration volume is completely filled,
capture a value of an operating parameter of the metering pump that characterizes one of a delivery rate of the metering pump and a delivery volume of the cleaning agent delivered by the metering pump, and
calibrate the metering pump by assigning a delivery rate calculated from the time required to fill the calibration volume and the size of the calibration volume or by assigning a size of the calibration volume as a delivery volume to the captured value of the operating parameter.

3. The device according to claim 2, wherein the metering pump is selected from a group consisting of a self-priming metering pump, a peristaltic pump, and an impeller meter, wherein the control and/or regulating apparatus is further configured to:
capture a rotary speed of the metering pump or a rotary speed of the impeller meter as the operating parameter of the metering pump while delivering the cleaning agent into the calibration volume, and
calibrate the metering pump in that the rotary speed captured while the cleaning agent is being delivered is assigned a calculated delivery rate of the metering pump by means of the size of the calibration volume and the time required to fill the calibration volume.

4. The device according to claim 2, wherein the metering pump is selected from a group consisting of a self-priming metering pump, a peristaltic pump, and an impeller meter, wherein the control and/or regulating apparatus is further configured to:
capture a number of rotations and/or cycles of the metering pump, and/or a number of rotations and/or cycles of the impeller meter, as the operating parameter of the metering pump during the delivery of cleaning agent into the calibration volume, and
perform a calibration of the metering pump by assigning a volume of the calibration volume as the delivery volume to the number of rotations and/or cycles cumulatively captured during the delivery of the cleaning agent.

5. The device according to claim 2, further comprising a first fill level sensor and a second fill level sensor provided with the measuring column, wherein the first fill level sensor is configured to capture a first level in the measuring column that corresponds to an empty state of the calibration volume, and wherein the second fill level sensor is configured to capture a second level in the measuring column that corresponds to a full state of the calibration volume, wherein the control and/or regulating apparatus is further configured to:
capture a first value of the operating parameter of the metering pump when a fill state in the measuring column is determined by the first fill level sensor that corresponds to or exceeds the first level,
capture a second value of the operating parameter of the metering pump when a fill state in the measuring column is determined by the second fill level sensor that corresponds to or exceeds the second level, and
determine a value for the operating parameter that characterizes the delivery amount needed to completely fill the calibration volume from the difference between the first and second value.

6. The device according to claim 5, wherein the control and/or regulating apparatus is further configured to lower the fill state of the measuring column below the level of the first level before a cleaning agent is delivered from the supply container into the calibration volume.

7. A method for operating the device according to claim 1, comprising the step of calibrating the device with the calibration device.

8. The method according to claim 7, comprising the steps of:
controlling the metering pump such that it delivers cleaning agent from the supply container into the calibration volume such that an initially empty calibration volume is completely filled,
capturing a value of an operating parameter of the metering pump that characterizes a delivery rate of the metering pump, or a delivery volume of the cleaning agent delivered by the metering pump, and
calibrating the metering pump by assigning a delivery rate calculated from the time required to fill the calibration volume and the size of the calibration volume, or by assigning a size of the calibration volume as a delivery volume to the captured value of the operating parameter.

9. The method according to claim 8, wherein the metering pump is selected from a group consisting of a self-priming metering pump, a peristaltic pump, and an impeller meter, the method further comprising the steps of:
capturing a rotary speed of the metering pump, and/or a rotary speed of the impeller meter as an operating parameter of the metering pump while delivering cleaning agent into the calibration volume, and
calibrating the metering pump such that the rotary speed captured while the cleaning agent is being delivered is assigned a calculated delivery rate of the metering pump by means of the size of the calibration volume and the time required to fill the calibration volume.

10. The method according to claim 8, wherein the metering pump is selected from a group consisting of a self-priming metering pump, a peristaltic pump, and an impeller meter, the method further comprising the steps of:
capturing a number of rotations and/or cycles of the metering pump, and/or a number of rotations and/or cycles of the impeller meter as an operating parameter of the metering pump during the delivery of cleaning agent into the calibration volume, and
calibrating the metering pump by assigning a volume of the calibration volume as the delivery volume to the number of rotations and/or cycles cumulatively captured during the delivery of the cleaning agent.

11. The method according to claim 7, wherein a first fill level sensor and a second fill level sensor are provided with the measuring column, the first fill level sensor being configured to capture a first level in the measuring column that corresponds to an empty state of the calibration volume, and the second fill level sensor being configured to capture a second level in the measuring column that corresponds to a fill state of the calibration volume, the method further comprising:
capturing a first value of the operating parameter of the metering pump when a fill state in the measuring column is determined by the first fill level sensor that corresponds to or exceeds the first level,
capturing a second value of the operating parameter of the metering pump when a fill state in the measuring column is determined by the second fill level sensor that corresponds to or exceeds the second level, and
determining a value for the operating parameter that characterizes the delivery amount needed to completely fill the calibration volume from the difference between the first and second value.

12. The method according to claim 11, further comprising the step of lowering the fill state of the measuring column below the level of the first level before cleaning agent is delivered from the supply container into the calibration volume.
